(19) United States
(12) Patent Application Publication
(10) Pub. No.: US 2025/0268566 A1
(43) Pub. Date: Aug. 28, 2025

(54) ULTRASONIC ENDOSCOPE

(71) Applicant: FUJIFILM Corporation, Tokyo (JP)

(72) Inventors: Kazushi FURUKAWA, Ashigarakami-gun (JP); Yasuhiko MORIMOTO, Ashigarakami-gun (JP); Karin HATTORI, Ashigarakami-gun (JP); Shozo IYAMA, Ashigarakami-gun (JP)

(73) Assignee: FUJIFILM Corporation, Tokyo (JP)

(21) Appl. No.: 19/054,896

(22) Filed: Feb. 16, 2025

(30) Foreign Application Priority Data
Feb. 28, 2024 (JP) 2024-028462

(51) Int. Cl. A61B 8/12; A61B 8/00; A61L 29/12; A61L 29/14

(52) U.S. Cl. CPC A61B 8/12; A61B 8/4483; A61L 29/126; A61L 29/14

(57) ABSTRACT

An ultrasonic endoscope includes: a distal end part which includes an ultrasound transmission and reception section and an imaging unit, the ultrasound transmission and reception section includes an ultrasonic oscillator and a backing material layer, and a glass transition temperature of the backing material layer is 45 degrees or lower.

### CROSS-REFERENCE TO RELATED APPLICATIONS

The present application claims priority under 35 U.S.C. § 119 to Japanese Patent Application No. 2024-028462 filed on Feb. 28, 2024. The above application is hereby expressly incorporated by reference, in its entirety, into the present application.

### BACKGROUND OF THE INVENTION

### 1. Field of the Invention

The present invention relates to an ultrasonic endoscope.

### 2. Description of the Related Art

JP2021-062170A, JP2011-176420A, and JP2005-177479A describe examples of physical properties of a backing material layer provided in an ultrasonic oscillator unit.

### SUMMARY OF THE INVENTION

An object of the present disclosure is to provide an ultrasonic endoscope that can be reduced in size.

An ultrasonic endoscope of one embodiment according to the technology of the present disclosure comprises: a distal end part including an ultrasound transmission/reception section and an imaging unit, in which the ultrasound transmission/reception section includes an ultrasonic oscillator and a backing material layer, and a glass transition temperature of the backing material layer is 45 degrees or lower.

According to the technology of the present disclosure, it is possible to achieve a reduction in size.

### BRIEF DESCRIPTION OF THE DRAWINGS

FIG. 1 is a schematic configuration diagram showing an example of an ultrasonic examination system 10 using an ultrasonic endoscope 12 according to an embodiment of the technology of the present disclosure.

FIG. 2 is a partially enlarged plan view showing a distal end part 40 shown in FIG. 1 and a vicinity thereof.

FIG. 3 is a cross-sectional view taken along line III-III shown in FIG. 2, and is a longitudinal cross-sectional view of the distal end part 40 taken along a center line thereof in a longitudinal axis direction.

FIG. 4 is a cross-sectional view taken along line IV-IV shown in FIG. 3, and is a transverse cross-sectional view taken along a center line of an arc structure of an ultrasonic oscillator array 50 of an ultrasonic observation part 36 of the distal end part 40.

FIG. 5 is a schematic view showing a cross section perpendicular to an axis of a signal cable 110.

FIG. 6 is a schematic view showing a cross section perpendicular to an axis of a cable 100.

FIG. 7 is an enlarged view of a portion including a substrate 60 and the cable 100.

FIG. 8 is a view showing a position of a filler 80 with a part of the cross section shown in FIG. 3 omitted.

FIG. 9 is a schematic cross-sectional view taken along line A-A of FIG. 7.

### DESCRIPTION OF THE PREFERRED EMBODIMENTS

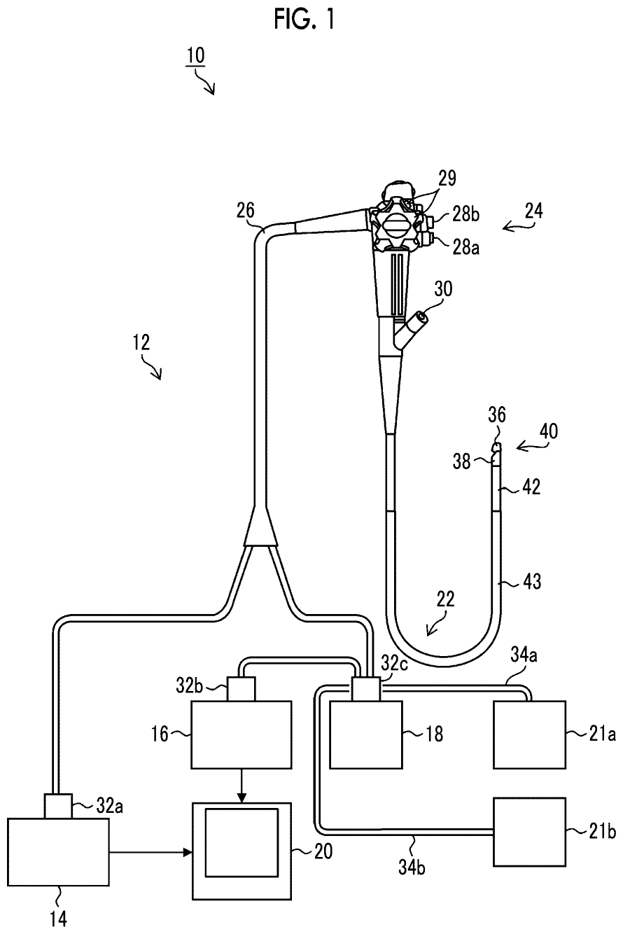

FIG. 1 is a schematic configuration diagram showing an example of an ultrasonic examination system 10 using an ultrasonic endoscope 12 according to an embodiment of the technology of the present disclosure. The ultrasonic examination system 10 comprises the ultrasonic endoscope 12, an ultrasound processor device 14 that generates an ultrasound image, an endoscope processor device 16 that generates an endoscopic image, a light source device 18 that supplies illumination light for illuminating an inside of a body cavity to the ultrasonic endoscope 12, a monitor 20 that displays the ultrasound image and the endoscopic image, a water supply tank 21a that stores cleaning water and the like, and a suction pump 21b that suctions an object to be suctioned in the body cavity.

The ultrasonic endoscope 12 has an insertion part 22 that is inserted into a body cavity of a subject, an operating part 24 that is consecutively provided in a proximal end part of the insertion part 22 and is used by an operator to perform an operation, and a universal cord 26 that has one end connected to the operating part 24.

In the operating part 24, an air/water supply button 28a that opens and closes an air/water supply pipe line (not shown) from the water supply tank 21a, and a suction button 28b that opens and closes a suction pipe line (not shown) from the suction pump 21b are provided side by side. In the operating part 24, a pair of angle knobs 29 and a treatment tool insertion port 30 are provided.

In the other end part of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasonic endoscope 12 is attachably and detachably connected to the ultrasound processor device 14, the endoscope processor device 16, and the light source device 18 via the connectors 32a, 32b, and 32c, respectively. The connector 32c comprises an air/water supply tube 34a that is connected to the water supply tank 21a, and a suction tube 34b that is connected to the suction pump 21b.

The insertion part 22 includes, in order from a distal end side, a distal end part 40 that has an ultrasonic observation part 36 and an endoscope observation part 38, a bendable part 42 that is consecutively provided on a proximal end side of the distal end part 40, and a soft part 43 that connects a proximal end side of the bendable part 42 and a distal end side of the operating part 24.

The bendable part 42 is remotely operated to be bent by rotationally operating the pair of angle knobs 29 provided in the operating part 24. With this, the distal end part 40 can be directed in a desired direction.

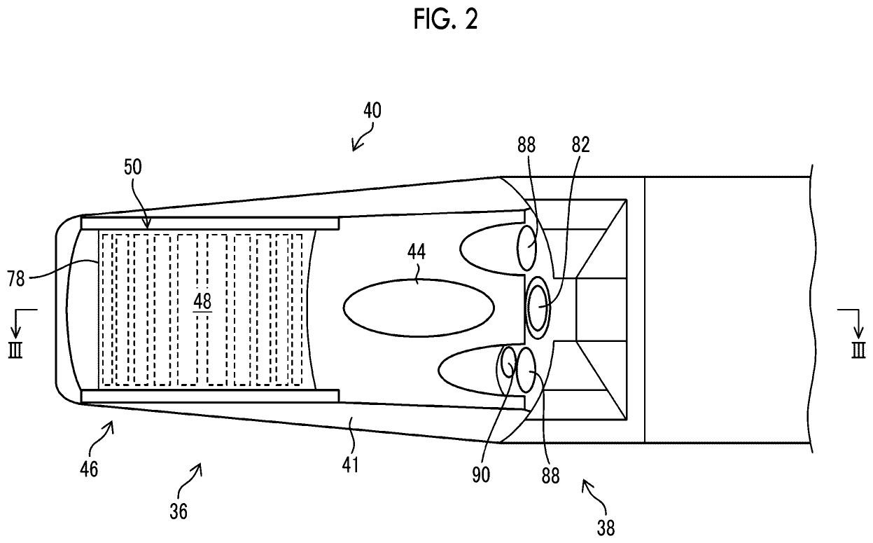

The ultrasound processor device 14 generates and supplies an ultrasound signal for causing an ultrasonic oscillator array 50 of an ultrasonic oscillator unit 46 (see FIG. 2) of the ultrasonic observation part 36 to generate ultrasonic waves. A vibration frequency of the ultrasonic oscillator 48 used in the ultrasonic endoscope 12 preferably has a center frequency of 5 MHz or more and 12 MHz or less. The ultrasound processor device 14 receives and acquires an echo signal reflected from an observation target site irradiated with the ultrasonic wave, by the ultrasonic oscillator array 50 and executes various kinds of signal processing on the acquired echo signal to generate an ultrasound image that is displayed on the monitor 20.

The endoscope processor device 16 receives and acquires a captured image signal acquired from the observation target site illuminated with illumination light from the light source device 18 in the endoscope observation part 38, and executes various kinds of processing on the acquired captured image signal to generate an endoscopic image that is displayed on the monitor 20.

In the example of FIG. 1, the ultrasound processor device 14 and the endoscope processor device 16 are configured with two devices (computers) provided separately. However, the present disclosure is not limited thereto, and both the ultrasound processor device 14 and the endoscope processor device 16 may be configured with one device.

In order to image an observation target site inside a body cavity using the endoscope observation part 38 to acquire a captured image signal, the light source device 18 generates illumination light, such as white light including light of three primary colors of red light, green light, and blue light or light of a specific wavelength. The illumination light propagates through a light guide (not shown) and the like in the ultrasonic endoscope 12 and is emitted from the endoscope observation part 38, and the observation target site inside the body cavity is illuminated with the illumination light.

The monitor 20 receives video signals generated by the ultrasound processor device 14 and the endoscope processor device 16 and displays the ultrasound image and the endoscopic image. In regard to the display of the ultrasound image and the endoscopic image, only one of these images may be appropriately switched and displayed on the monitor 20 or both the images may be displayed simultaneously.

In the present embodiment, although the ultrasound image and the endoscopic image are displayed on one monitor 20, a monitor for ultrasound image display and a monitor for endoscopic image display may be provided separately. In addition, the ultrasound image and the endoscopic image may be displayed in a display form other than the monitor 20, for example, in a form of being displayed on a display of a terminal carried by the operator.

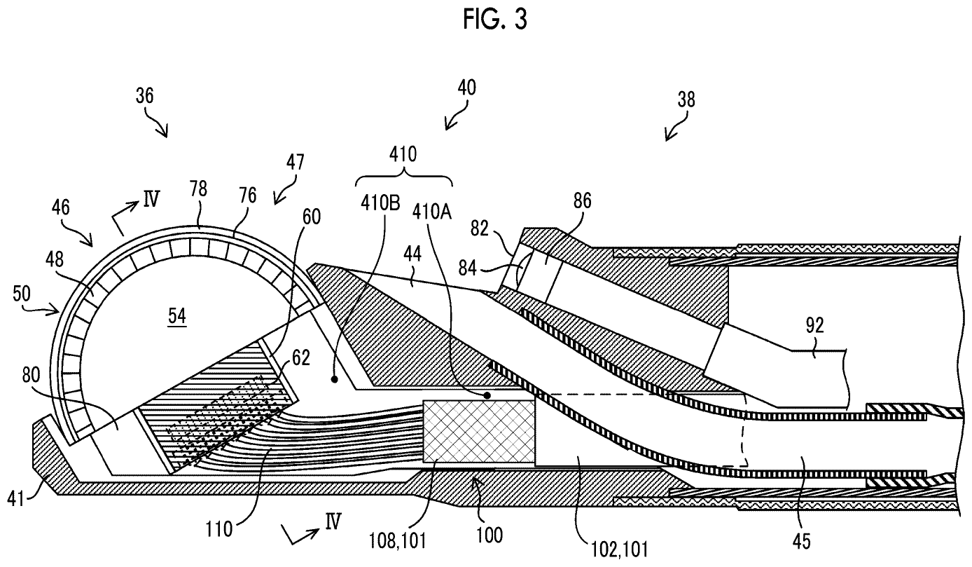

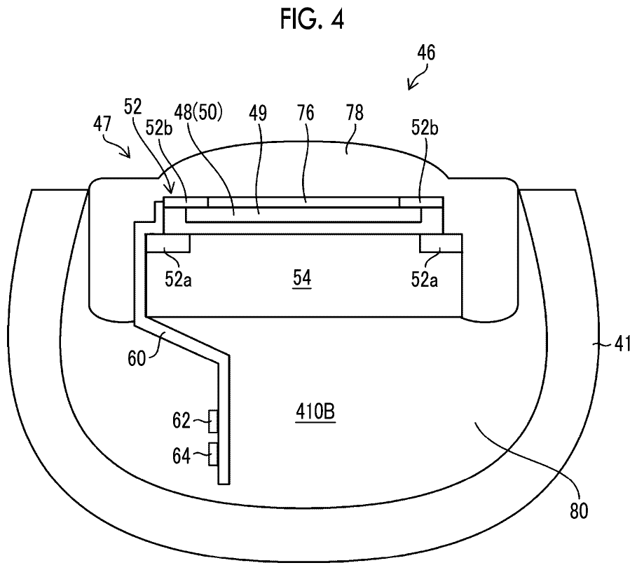

Next, a configuration of the distal end part 40 will be described with reference to FIGS. 2 to 4. FIG. 2 is a partially enlarged plan view showing the distal end part 40 shown in FIG. 1 and a vicinity thereof. FIG. 3 is a cross-sectional view taken along line III-III shown in FIG. 2, and is a longitudinal cross-sectional view of the distal end part 40 taken along a center line thereof in a longitudinal axis direction. FIG. 4 is a cross-sectional view taken along line IV-IV shown in FIG. 3, and is a transverse cross-sectional view taken along a center line of an arc structure of the ultrasonic oscillator array 50 of the ultrasonic observation part 36 of the distal end part 40.

As shown in FIGS. 2 and 3, in the distal end part 40, the ultrasonic observation part 36 for acquiring the ultrasound image is mounted on the distal end side, and the endoscope observation part 38 for acquiring the endoscopic image is mounted on the proximal end side. In the distal end part 40, a treatment tool outlet port 44 is provided between the ultrasonic observation part 36 and the endoscope observation part 38.

The endoscope observation part 38 comprises an observation window 82, an objective lens 84, an imaging clement 86, an illumination window 88, a cleaning nozzle 90, a wiring cable 92, and the like. The observation window 82, the objective lens 84, the imaging element 86, and the illumination window 88 constitute an imaging unit.

The treatment tool outlet port 44 is connected to a treatment tool channel 45 that is inserted into the insertion part 22. A treatment tool (not shown) inserted from the treatment tool insertion port 30 of FIG. 1 is led out from the treatment tool outlet port 44 into the body cavity via the treatment tool channel 45.

As shown in FIGS. 2 to 4, the ultrasonic observation part 36 comprises the ultrasonic oscillator unit 46 constituting an ultrasound transmission/reception section, an exterior member 41 that holds the ultrasonic oscillator unit 46, and a cable 100 that is electrically connected to the ultrasonic oscillator unit 46 via a substrate 60. The cable 100 forms an elongated shape extending along a longitudinal axis direction of the insertion part 22, and is provided to extend to the connector 32a.

The exterior member 41 is made of a hard member, such as hard resin, and constitutes a part of the distal end part 40. In the exterior member 41, an accommodation space 410 that penetrates the exterior member 41 in the longitudinal axis direction of the insertion part 22 is provided. The accommodation space 410 includes a first space 410A on the proximal end side and a second space 410B on the distal end side, which is larger than the first space 410A. A part of the ultrasonic oscillator unit 46, the substrate 60, and a distal end side of the cable 100 are accommodated in the accommodation space 410. The accommodation space 410 constitutes an accommodation portion that accommodates the ultrasonic oscillator unit 46 and the cable 100.

The ultrasonic oscillator unit 46 has the ultrasonic oscillator array 50 consisting of a plurality of the ultrasonic oscillators 48, an electrode 52 provided on an end part side of the ultrasonic oscillator array 50 in a width direction (direction perpendicular to the longitudinal axis direction of the insertion part 22), a backing material layer 54 that supports each ultrasonic oscillator 48 from a lower surface side, and the substrate 60 that is disposed along a side surface of the backing material layer 54 in the width direction and is connected to the electrode 52.

As long as the substrate 60 can electrically connect the plurality of ultrasonic oscillators 48 and the cable 100, in particular, a structure thereof is not limited.

It is preferable that the substrate 60 is configured with, for example, a wiring substrate, such as a flexible substrate (flexible printed substrate (also referred to as a flexible printed circuit (FPC)) having flexibility, a printed wiring circuit substrate (also referred to as a printed circuit board (PCB)) made of a rigid substrate having high rigidity with no flexibility, or a printed wiring substrate (also referred to as a printed wired board (PWB)).

The ultrasonic oscillator unit 46 has an acoustic matching layer 76 laminated on the ultrasonic oscillator array 50, and an acoustic lens 78 laminated on the acoustic matching layer 76. The ultrasonic oscillator unit 46 is configured as a laminate 47 having the acoustic lens 78, the acoustic matching layer 76, the ultrasonic oscillator array 50, and the backing material layer 54.

The ultrasonic oscillator array 50 is configured with a plurality of rectangular parallelepiped ultrasonic oscillators 48 arranged in a convex arc shape outward. The ultrasonic oscillator array 50 is, for example, an array of 48 channels to 192 channels consisting of 48 to 192 ultrasonic oscillators 48. Each ultrasonic oscillator 48 has a piezoelectric body 49.

The ultrasonic oscillator array 50 has the electrode 52. The electrode 52 has an individual electrode 52a individually and independently provided for each ultrasonic oscillator 48, and an oscillator ground 52b that is a common electrode common to all the ultrasonic oscillators 48. In FIG. 4, a plurality of the individual electrodes 52a are disposed on lower surfaces of end parts of the plurality of ultrasonic oscillators 48, and the oscillator ground 52b is disposed on upper surfaces of the end parts of the ultrasonic oscillators 48.

The substrate 60 has 48 to 192 wirings (not shown) that are electrically connected to the individual electrodes 52a of 48 to 192 ultrasonic oscillators 48, respectively, and a plurality of electrode pads 62 that are connected to the ultrasonic oscillators 48 through the wirings, respectively.

The ultrasonic oscillator array 50 has a configuration in which a plurality of the ultrasonic oscillators 48 are arranged at a predetermined pitch in a one-dimensional array shape as an example. The ultrasonic oscillators 48 constituting the ultrasonic oscillator array 50 are arranged at regular intervals in a convexly curved shape along the longitudinal axis direction of the insertion part 22, and are sequentially driven based on a driving signal input from the ultrasound processor device 14 (see FIG. 1). With this, convex electronic scanning is performed with a range in which the ultrasonic oscillators 48 shown in FIG. 2 are arranged, as a scanning range.

The acoustic matching layer 76 is provided for taking acoustic impedance matching between the subject and the ultrasonic oscillators 48.

The acoustic lens 78 is provided for converging the ultrasonic waves emitted from the ultrasonic oscillator array 50 toward the observation target site. The acoustic lens 78 is formed of, for example, a silicone-based resin (such as a millable silicone rubber or a liquid silicone rubber), a butadiene-based resin, a polyurethane-based resin, or the like. In the acoustic lens 78, powder, such as titanium oxide, alumina, or silica, is mixed as necessary. With this, the acoustic lens 78 can take acoustic impedance matching between the subject and the ultrasonic oscillators 48 in the acoustic matching layer 76, and can increase a transmittance of the ultrasonic waves.

As shown in FIGS. 3 and 4, the backing material layer 54 is disposed on an inner side with respect to an arrangement surface of a plurality of the ultrasonic oscillators 48, that is, a rear surface (lower surface) of the ultrasonic oscillator array 50. The backing material layer 54 is composed of a layer of a member made of a backing material. The backing material layer 54 has a role of mechanically and flexibly supporting the ultrasonic oscillator array 50 and attenuating ultrasonic waves propagated to the backing material layer 54 side among ultrasound signals oscillated from the plurality of ultrasonic oscillators 48 or reflected and propagated from an observation target. In consideration of a reduction in diameter of the ultrasonic endoscope 12 and attenuation performance of the ultrasonic waves, a thickness of the backing material layer 54 is preferably 0.5 mm or more and 1.5 mm or less.

The substrate 60 shown in FIG. 4 has a plurality of the electrode pads 62 that are electrically connected to the plurality of individual electrodes 52a at one end, and a ground electrode pad 64 that is electrically connected to the oscillator ground 52b. In FIG. 4, the cable 100 is omitted.

Electrical bonding of the substrate 60 and the individual electrodes 52a can be established by, for example, a resin material having conductivity. Examples of the resin material include an anisotropic conductive film (ACF) or an anisotropic conductive paste (ACP) obtained by mixing fine conductive particles with a thermosetting resin and forming the mixture into a film.

As another resin material, for example, a resin material in which conductive fillers, such as metallic particles, are dispersed into a binder resin, such as epoxy or urethane, and the fillers form a conductive path after adhesion may be used. Examples of this resin material include a conductive paste, such as a silver paste.

As shown in FIG. 3, the cable 100 includes a plurality of signal cables 110 and a tubular covering portion 101 that bundles and covers the plurality of signal cables 110.

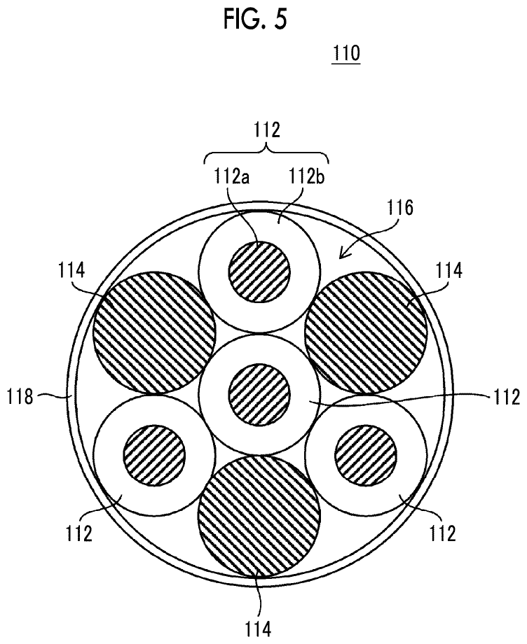

FIG. 5 is a schematic view showing a cross section perpendicular to an axis of the signal cable 110. In the example of FIG. 5, the signal cable 110 is a non-coaxial cable. The signal cable 110 includes a plurality of signal lines 112 and a plurality of ground lines 114. The signal line 112 is composed of, for example, a conductor 112a and an insulating layer 112b that covers an outer peripheral surface of the conductor 112a.

The conductor 112a is composed of, for example, an element wire of copper, a copper alloy, or the like. The element wire is subjected to, for example, a plating treatment, such as tin plating or silver plating. The conductor 112a has, for example, a diameter of 0.03 mm to 0.04 mm. The insulating layer 112b can be made of, for example, a resin material, such as fluorinated-ethylene-propylene (FEP) or perfluoroalkoxy (PFA). The insulating layer 112b has, for example, a thickness of 0.015 mm to 0.025 mm.

The ground line 114 is composed of, for example, a conductor having the same diameter as the signal line 112. The ground line 114 is composed of an element wire of copper or a copper alloy or a twisted wire obtained by twisting a plurality of element wires of copper or a copper alloy.

A first signal line bundle 116 is configured by twisting together a plurality of the signal lines 112 and a plurality of the ground lines 114.

The signal cable 110 comprises a first shield layer 118 that bundles and covers the first signal line bundle 116. The first shield layer 118 can be made of an insulating film obtained by laminating metal foils via an adhesive, or the like. The insulating film is made of a polyethylene terephthalate (PET) film or the like. The metal foil is made of an aluminum foil, a copper foil, or the like.

The signal cable 110 is shielded by the first shield layer 118 with the plurality of signal lines 112 as one set.

The first signal line bundle 116 is configured by twisting seven lines including four signal lines 112 and three ground lines. One signal line 112 of the four signal lines 112 is disposed at a center. The remaining three signal lines 112 and three ground lines 114 are disposed adjacently in a periphery of the signal line 112 at the center. However, the number of signal lines 112, the number of ground lines 114, and arrangement thereof in the first signal line bundle 116 are not limited to a structure of FIG. 5. Each conductor 112a included in the signal cable 110 is electrically connected to any of the electrode pads 62 of the substrate 60.

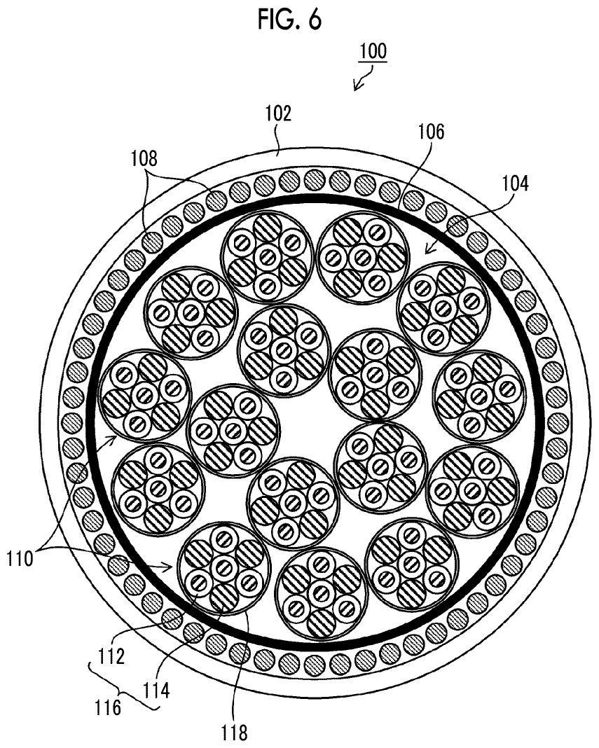

FIG. 6 is a schematic view showing a cross section perpendicular to an axis of the cable 100. In the example of FIG. 6, the cable 100 comprises a plurality of the signal cables 110, a tubular resin layer 106 that bundles and covers the plurality of signal cables 110, a tubular second shield layer 108 that is provided along an outer peripheral surface of the resin layer 106 and covers the outer peripheral surface of the resin layer 106, and a tubular outer cover 102 that is provided along an outer peripheral surface of the second shield layer 108 and covers the outer peripheral surface of the second shield layer 108. The covering portion 101 is configured with the resin layer 106, the second shield layer 108, and the outer cover 102.

The outer cover 102 can be made of a fluorine-based resin material, such as PFA, FEP, an ethylene-tetrafluoroethylene copolymer (ETFE), polyvinyl chloride (PVC), or the like, which is extrusion-molded. The outer cover 102 constitutes an outermost peripheral surface of the cable 100. It is preferable that an outer surface of the outer cover 102 has a high smoothness in order to reduce friction with other contents (an air/water supply tube, a suction tube, a pulling wire, or the like) inside the ultrasonic endoscope 12 and to improve robustness.

The resin layer 106 can be formed of, for example, the above-described fluorine-based resin material or a tape made of a resin.

It is preferable that an outer surface of the second shield layer 108 has a smoothness lower than the smoothness of the outer surface of the outer cover 102. The smoothness can be defined by, for example, an average surface roughness. The second shield layer 108 is, for example, a metal mesh shield formed by braiding a plurality of element wires. The element wire is made of a copper wire, a copper alloy wire, or the like subjected to a plating treatment (tin plating or silver plating). The resin layer 106 and the second shield layer 108 constitute a first covering member that bundles and covers the plurality of signal cables 110. The resin layer 106 is not essential to the cable 100 and may be omitted. The outer cover 102 constitutes a second covering member that covers the first covering member.

The second shield layer 108 is provided in a concentric circular shape with the resin layer 106 on an outer periphery of the resin layer 106, and surrounds and covers the outer peripheral surface of the resin layer 106 in a range of 360 degrees in a circumferential direction. The outer cover 102 is provided in a concentric circular shape with the second shield layer 108 on an outer periphery of the second shield layer 108, and surrounds and covers the outer peripheral surface of the second shield layer 108 in a range of 360 degrees in a circumferential direction.

In the example of FIG. 6, the cable 100 includes 16 signal cables 110 and includes 64 signal lines 112. The number of signal cables 110 and the number of signal lines 112 are not limited to these numerical values.

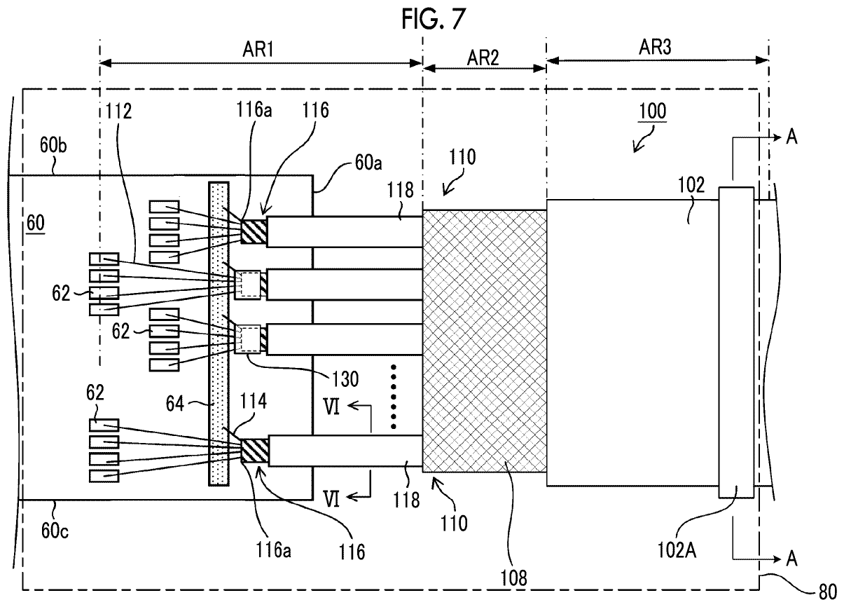

FIG. 7 is an enlarged view of a portion including the substrate 60 and the cable 100. As shown in FIG. 7, the substrate 60 has a plurality of the electrode pads 62 disposed along a side 60a on the proximal end side, and the ground electrode pad 64 disposed between the plurality of electrode pads 62 and the side 60a. The ground electrode pad 64 is disposed in parallel to the side 60a.

The cable 100 is disposed at a position facing the side 60a of the substrate 60. The electrode pad 62 and the signal line 112 of the signal cable 110 are electrically connected to each other. The signal cable 110 is disposed in parallel to a side 60b and a side 60c that are perpendicular to the side 60a. However, a positional relationship between the substrate 60 and the signal cable 110 is not particularly limited.

As shown in FIGS. 3 and 7, the cable 100 has a first region AR1 in which the covering portion 101 is peeled off and the signal cable 110 is partially exposed, on the distal end side thereof. The cable 100 has a second region AR2 in which the outer cover 102 is peeled off and the second shield layer 108 is partially exposed, on the proximal end side with respect to the first region AR1. The cable 100 has a third region AR3 in which the outer cover 102 is exposed, on the proximal end side with respect to the second region AR2. As described above, in the accommodation space 410, the cable 100 comprises, in order from the ultrasonic oscillator unit 46 side, the first region AR1 in which the signal cable 110 is exposed, the second region AR2 in which the second shield layer 108 is exposed, and the third region AR3 in which the outer cover 102 is exposed.

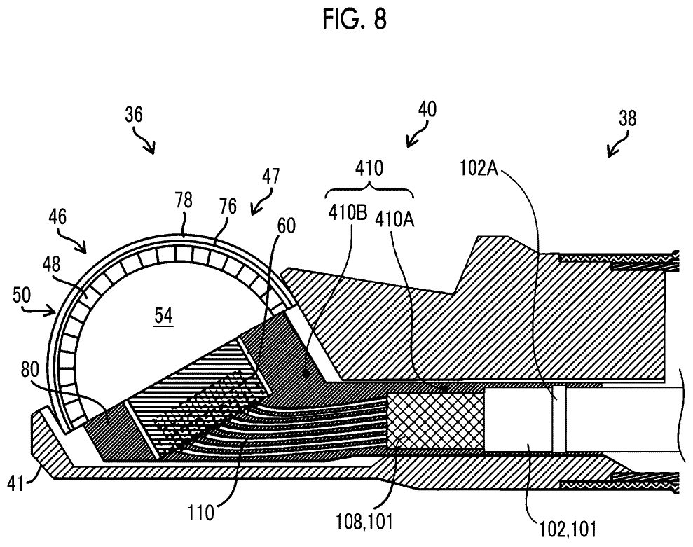

In the accommodation space 410 shown in FIG. 3, in a gap between the exterior member 41 and the ultrasonic oscillator unit 46, the substrate 60, and the distal end side of the cable 100 (a portion other than the cable 100 in the first space 410A and a portion other than the ultrasonic oscillator unit 46, the substrate 60, and the cable 100 in the second space 410B), a filler 80 is provided to fill the gap. FIG. 8 is a view showing a position of the filler 80 with a part of the cross section shown in FIG. 3 omitted.

The filler 80 mainly has a role of fixing the substrate 60, the signal cable 110, and various wiring portions. It is preferable that an acoustic impedance of the filler 80 matches an acoustic impedance of the backing material layer 54 with a certain accuracy or higher such that the ultrasound signals propagated from the ultrasonic oscillator array 50 to the backing material layer 54 side are not reflected at a boundary surface between the filler 80 and the backing material layer 54. It is preferable that the filler 80 is made of a member having heat radiation properties to increase an efficiency for radiating heat generated in the plurality of ultrasonic oscillators 48. In a case where the filler 80 has heat radiation properties, the filler 80 receives heat from the backing material layer 54, the substrate 60, the signal cable 110, and the like, and thus a heat radiation efficiency can be improved. A material of the filler 80 is not particularly limited, and for example, a silicone resin, rubber, or the like is used.

As shown in FIG. 7, the filler 80 fills a gap between an inner surface of the exterior member 41 and the first region AR1, the second region AR2, and the third region AR3, and is in contact with the first region AR1, the second region AR2, and the third region AR3.

With such a configuration, an anchor effect can be obtained by the filler 80 being embedded in a level difference at a boundary portion between the first region AR1 and the second region AR2, a level difference at a boundary portion between the second region AR2 and the third region AR3, or the like. As a result, a fixing force of various members by the filler 80 can be increased, and a durability of the ultrasonic endoscope 12 can be improved.

In addition, in a case where a smoothness of an outer surface of the second region AR2 is lower than a smoothness of an outer surface of the third region AR3, the filler 80 can also be embedded in an unevenness of the outer surface of the second region AR2 to obtain an anchor effect. As a result, the durability of the ultrasonic endoscope 12 can be further improved. In the present embodiment, the second region AR2 and the third region AR3 are disposed in the first space 410A, which is relatively narrow, in the accommodation space 410. Therefore, a volume of a gap between the exterior member 41 and the second region AR2 and the third region AR3 is small, and there is little room for the filler 80 to enter. Even in such a configuration, since the smoothness of the second shield layer 108 is low, a sufficient fixing force can be secured even with a small amount of the filler 80.

As shown in FIGS. 7 and 8, a protrusion 102A that protrudes in a radial direction of the cable 100 is provided on an outer surface (surface of the outer cover 102) of a portion of the third region AR3 of the cable 100, which is disposed in the first space 410A.

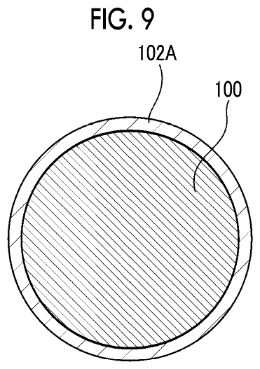

FIG. 9 is a schematic cross-sectional view taken along line A-A of FIG. 7. In FIG. 9, a cross section of the cable 100 is shown in a simplified manner. As shown in FIG. 9, the protrusion 102A is formed of an annular member provided on an entire circumference of an outer peripheral surface of the outer cover 102 of the cable 100 along the outer peripheral surface. An outer shape of the annular member is not particularly limited, and a true circle, an ellipse, a polygon, or the like can be adopted. The protrusion 102A may be integrally formed with the outer cover 102 of the cable 100, but is preferably a separate body from the cable 100. For example, by forming the protrusion 102A with a metal ring or the like, the cable 100 can be tightened by the protrusion 102A from an outer peripheral side thereof. As a result, it is possible to prevent the outer cover 102 from moving in an axial direction with respect to the second shield layer 108 in the first space 410A. In addition, the filler 80 is embedded in the protrusion 102A so that an anchor effect can be obtained, and the durability of the ultrasonic endoscope 12 can be further improved.

The protrusion 102A does not have to be provided on the entire circumference of the outer peripheral surface of the outer cover 102 of the cable 100 along the outer peripheral surface. For example, the protrusion 102A may have a C-shape as shown in FIG. 10. By forming the protrusion 102A in a C-shape, in a case where the cable 100 and the protrusion 102A are separate bodies, it is easy to mount the protrusion 102A on the cable 100. In addition, the anchor effect can be enhanced by the filler 80 being embedded between both circumferential ends of the C-shaped protrusion 102A. The C-shaped protrusion 102A shown in FIG. 10 is an example of an annular member.

In addition, as long as the purpose is to obtain the anchor effect, the protrusion 102A does not have to be formed of an annular member, and any shape can be adopted. By forming the protrusion 102A with an annular member, the anchor effect can be obtained while tightening the cable 100 as described above. A plurality of the protrusions 102A may be provided along an axial direction of the cable 100. As a result, the anchor effect can be further enhanced.

As shown in FIG. 7, the substrate 60 and the first signal line bundle 116 are fixed by a fixing part 130, and a relative position between the substrate 60 and each first signal line bundle 116 is fixed. The fixing part 130 fixes the substrate 60 and the first signal line bundle 116 in a state of overlapping the substrate 60. The first signal line bundle 116 composed of a twisted wire of the plurality of signal lines 112 and the plurality of ground lines 114 is disentangled into individual signal lines 112 at a distal end 116a. Each disentangled signal line 112 is electrically connected to the electrode pad 62 disposed on the substrate 60. The distal end 116a is a start position where each signal line 112 is disentangled. In addition, the fixing part 130 is omitted in some of the first signal line bundles 116 for ease of understanding. A connection region between the substrate 60 and the signal cable 110 as described above is also covered and fixed by the above-described filler 80.

Next, preferred configurations of the backing material layer 54 and the filler 80 will be described.

### Preferred Physical Properties of Backing Material Layer

A glass transition temperature of the backing material layer 54 is preferably 45 degrees or lower.

A temperature of an environment in which the ultrasonic endoscope 12 is placed can change from a temperature of a storage place to a temperature (about 45 degrees) inside the subject. The ultrasonic endoscope 12 may be placed in an environment of a temperature range of, for example, 5 degrees or higher and 45 degrees or lower, depending on a storage environment. In a case where the glass transition temperature of the backing material layer 54 is equal to or lower than 45 degrees which is the temperature inside the subject, physical properties of the backing material layer 54 changed in a case where the ultrasonic endoscope 12 is inserted into the subject. For example, in the backing material layer 54, a state in which movement of molecules is intensified is achieved, and a state in which external energy (ultrasonic wave) is easily consumed as kinetic energy (heat) of the molecules is achieved. As a result, even in a case where the thickness of the backing material layer 54 is reduced to reduce the diameter (size) of the ultrasonic endoscope 12, the attenuation performance of the ultrasonic waves by the backing material layer 54 can be increased during insertion of the ultrasonic endoscope 12 into the subject. In addition, since the backing material layer 54 is softened, stress in a case where a thermal load is applied to the ultrasonic endoscope 12 is reduced, and thus it is possible to prevent breakage or the like and to increase the durability.

On the other hand, in a state in which the ultrasonic endoscope 12 is outside the subject, the ultrasonic endoscope 12 is placed in a temperature environment lower than the glass transition temperature of the backing material layer 54. In this temperature environment, the backing material layer 54 has sufficient hardness. Therefore, the durability can be improved by enhancing an impact resistance and the like in a case where the ultrasonic endoscope 12 is stored. In a case where the ultrasonic endoscope 12 is inserted into the subject, the backing material layer 54 is slightly softened, but an interior wall of an organ of the subject is also soft, so that an influence on the durability is slight.

Considering that the ultrasonic endoscope 12 is stored indoors, a lower limit value of the glass transition temperature of the backing material layer 54 is about 5 degrees. However, it is better that the lower limit value is set to about 10 degrees in consideration of a more practical storage environment. In addition, in consideration of the storage in an indoor place in an air-conditioned environment, it is advisable to set the lower limit value to about 20 degrees. In addition, considering that there may be variations in temperature inside the subject, an upper limit value of the glass transition temperature of the backing material layer 54 is preferably set to 40 degrees. In consideration of a practical use environment of the ultrasonic endoscope 12 and ease of manufacturing the backing material layer 54, the glass transition temperature of the backing material layer 54 is more preferably 25 degrees or higher and 35 degrees or lower.

The material constituting the backing material layer 54 is not particularly limited, but for example, it is preferable that the backing material layer 54 is configured to include at least one kind of a polyurea resin, an epoxy resin having a polyurethane structure, or an epoxy resin having a polyetheramine structure. By being configured to include these resins, the stress caused by the thermal load can be sufficiently reduced. From the viewpoint of further improving the workability, it is preferable that the resin included in the backing material layer 54 includes a polyurea resin.

As the resin included in the backing material layer 54, a resin having a loss tangent of 0.06 or more in a range of 0° C. to 50° C. and a loss tangent of less than 1.50 in a range of −20° C. to 110° C. can be preferably used. It is preferable that a storage elastic modulus of the backing material layer 54 in a range of 0° C. to 50° C. is 1,000 MPa or more in a case where the content of the resin in the backing material layer 54 is 25% to 50% by volume.

Hereinafter, details of the preferred resin included in the backing material layer 54 will be described.

### Polyurea Resin

The polyurea resin can be obtained by a reaction between a polyisocyanate compound and a polyamine compound.

As the polyisocyanate compound, any polyisocyanate compound having two or more isocyanato groups can be used without particular limitation. The polyisocyanate compound may be any of an aliphatic isocyanate compound (a compound in which an isocyanato group is bonded to an aliphatic chain or an aliphatic ring) or an aromatic isocyanate compound (a compound in which an isocyanato group is bonded to an aromatic ring), or may be a mixture thereof. The polyisocyanate compound may have a ring structure. From the viewpoint of low reactivity and a long pot life during production of a cured substance, an aliphatic polyisocyanate compound is preferable as the polyisocyanate compound, and from the viewpoint of further improving ultrasonic wave attenuation properties, it is preferable that the polyisocyanate compound includes an aliphatic polyisocyanate compound having an aromatic ring and an aromatic polyisocyanate compound.

The polyamine compound can be used without particular limitation as long as it is a polyamine compound having two or more amino groups, and a polyamine compound generally used as a curing agent for an epoxy resin is preferably used. The polyamine compound may be any of an aliphatic polyamine compound (a chain-like aliphatic polyamine compound in which an amino group is bonded to an aliphatic chain or a cyclic aliphatic polyamine compound in which an amino group is bonded to an aliphatic ring) or an aromatic polyamine compound (a compound in which an amino group is bonded to an aromatic ring), and may be a mixture thereof. Among these, an aliphatic polyamine compound is preferable because it has excellent reactivity. The polyamine compound may have a ring structure. In addition to a nitrogen atom, a heteroatom such as an oxygen atom may be included. From the viewpoint of further improving the ultrasonic wave attenuation properties and the workability, the polyamine compound preferably includes an aliphatic polyamine compound having an aromatic ring and a chain-like aliphatic polyamine compound having no aromatic ring.

### Epoxy Resin Having Polyurethane Structure

The epoxy resin having a polyurethane structure can be used without particular limitation as long as it is an epoxy resin having a polyurethane structure and an epoxy group. As a commercially available epoxy resin having a polyurethane structure, for example, an epoxy resin having a polyurethane structure with a number average molecular weight of 200 to 20,000 is generally used. A viscosity of the epoxy resin having a polyurethane structure at 25° C. is not particularly limited, and is, for example, preferably 200 to 200,000 mPa·sec and more preferably 600 to 30,000 mPa·sec. The viscosity is a value measured under conditions of 25° C. and a shear rate of 0.01/sec.

As the curing agent for reacting with the epoxy resin having a polyurethane structure, a polyamine or an acid anhydride can be used, but it is preferable to use a polyamine as the curing agent. The polyamine compound to be reacted with the epoxy resin having a polyurethane structure can be used without particular limitation as long as it is a polyamine compound having two or more amino groups, and a polyamine compound generally used as a curing agent for an epoxy resin is preferably used.

### Epoxy Resin Having Polyetheramine Structure

As the epoxy resin having a polyetheramine structure, a reaction cured product of an epoxy resin and a polyamine compound having two or more amino groups can be used without particular limitation as long as it has a polyether structure.

The epoxy resin having a polyetheramine structure can be obtained by any of a reaction between an epoxy resin having a polyether structure and a polyamine compound having no polyether structure, a reaction between an epoxy resin having no polyether structure and a polyamine compound having a polyether structure, or a reaction between an epoxy resin having a polyether structure and a polyamine compound having a polyether structure. In general, the polyether structure of the reaction cured product obtained in this way is generally a polyether structure having a number average molecular weight of 200 to 6,000.

Among these, a reaction cured product of an epoxy resin having a polyether structure and a polyamine compound having no polyether structure, or a reaction cured product of an epoxy resin having no polyether structure and a polyamine compound having a polyether structure is preferable, and from the viewpoint of exhibiting a more preferable viscosity as a curable resin composition, a reaction cured product of an epoxy resin having no polyether structure and a polyamine compound having a polyether structure is more preferable.

As a commercially available epoxy resin having a polyether structure, for example, an epoxy resin having a polyether structure with a number average molecular weight of 200 to 6,000 is generally used, and specific examples thereof include the following epoxy resins. From the viewpoint of excellent mechanical strength, it is preferable that the epoxy resin has a bisphenol structure. Examples of the epoxy resin having a polyether structure include a bisphenol A epoxy resin, a bisphenol F epoxy resin, a bisphenol E epoxy resin, and a novolac epoxy resin. From the viewpoint of excellent mechanical strength of a cured product, a bisphenol A epoxy resin is preferable.

The polyamine compound having a polyether structure can be used without particular limitation as long as it is a polyamine compound having two or more amino groups, and a polyamine compound generally used as a curing agent for an epoxy resin is preferably used. As a commercially available polyamine compound having a polyether structure, for example, a polyamine compound having a polyether structure with a number average molecular weight of 200 to 6,000 is generally used.

### Content of Resin in Backing Material Layer

A content of a resin in the backing material layer 54 is 25% to 50% by volume, and preferably 30% to 50% by volume. A content of a reaction cured product of at least one kind of the polyurea resin, the epoxy resin having a polyurethane structure, or the epoxy resin having a polyetheramine structure, which are resins included in the backing material layer 54, is not particularly limited as long as the effects of the technology of the present disclosure are exhibited. For example, the content can be 15% by volume or more, and is preferably 20% by volume or more, more preferably 30% by volume or more, still more preferably 50% by volume or more, and particularly preferably 70% by volume or more. It is also preferable that all the resins included in the backing material layer 54 are composed of at least one kind of the polyurea resin, the epoxy resin having a polyurethane structure, or the epoxy resin having a polyetheramine structure.

The backing material layer 54 is preferably configured using at least one kind of the polyurea resin, the epoxy resin having a polyurethane structure, or the epoxy resin having a polyetheramine structure as a base material, and including a heat radiation filler. The backing material layer 54 includes thermally conductive particles as the heat radiation filler, and thus a thermal conductivity can be increased. By increasing the thermal conductivity of the backing material layer 54, heat generated in the ultrasonic oscillator unit 46 can be transmitted to a heat radiation structure (not shown), and accumulation of the heat in the distal end part 40 can be prevented. As a result, a thermal load on the backing material layer 54 is reduced, and stress due to the thermal load can be further reduced.

As long as the thermally conductive particles have thermally conductive properties, any of inorganic particles or organic particles may be used. In order to increase the thermally conductive properties of the backing material layer 54, a thermal conductivity per unit weight of the backing material layer 54 is preferably 30 W/m·K or more and more preferably 60 W/m·K or more. Since the ultrasonic endoscope 12 is inserted into a body, it is preferable that the thermally conductive particles are a safe material having no toxicity or the like and are stable against a use environment such as hygroscopicity. In addition, in order to increase the attenuation properties, it is preferable that a density of the thermally conductive particles is high. Since the thermally conductive particles are disposed in the vicinity of a circuit, a material having low or no conductivity that does not cause a short circuit failure is preferable.

A shape of the thermally conductive particle is not particularly limited, and various shapes such as an amorphous shape, a spherical shape, a fibrous shape, a branched fibrous shape, and a flat plate shape are used. In a case where the shape is a spherical shape, it is possible to increase a filling rate, which is preferable. In a case where the shape is an anisotropic shape such as a fibrous shape or a flat plate shape, it is possible to increase a particle contact and improve heat radiation properties, which is preferable. In a case of the amorphous particles, the ultrasonic waves can be randomly reflected, which is preferable from the viewpoint of improving the ultrasonic wave attenuation properties of the backing material layer 54.

Examples of the thermally conductive particles include aluminum oxide, tungsten oxide, silicon carbide, tungsten carbide, silicon nitride, boron nitride, and aluminum nitride. In particular, from the viewpoint of high thermal conductivity and high insulating properties, a nitride is preferable. The thermally conductive particles may include one kind of these thermally conductive materials or may include two or more kinds thereof. A surface of the thermally conductive particle may be subjected to a surface treatment in order to easily disperse in the resin.

A particle diameter of the thermally conductive particle is not particularly limited. From the viewpoint of maintaining the mechanical strength of the backing material layer 54 high while suppressing the viscosity of the curable resin composition included in the backing material layer 54 to be low, for example, the particle diameter of the thermally conductive particle is preferably 1 to 300 μm, more preferably 5 to 100 μm, and still more preferably 8 to 30 μm. The “particle diameter” of the thermally conductive particle is a number average particle diameter.

A proportion of the thermally conductive particles in a total amount of components other than the above-described resins in the backing material layer 54 is preferably 50% by volume or more, more preferably 60% by volume or more, and still more preferably 65% by volume or more. It is also preferable that all the components other than the above-described resins in the backing material layer 54 are thermally conductive particles. For example, the content of the thermally conductive particles in the backing material layer 54 is preferably 30% to 60% by volume, more preferably 30% to 55% by volume, and still more preferably 30% to 50% by volume.

The backing material layer 54 may contain other components in addition to the above-described resins and thermally conductive particles. Hollow particles may be included as the other components. By including the hollow particles, the ultrasonic wave attenuation properties can be further improved. As the hollow particles, hollow particles commonly used for exhibiting the effect of improving acoustic wave attenuation properties or ultrasonic wave attenuation properties can be used without particular limitation. Any of hollow glass particles or hollow resin particles may be used, and it is preferable to use hollow resin particles.

Preferred examples of the hollow particles include glass balloons, hollow silica, cenolite, phenolic resin microballoons, urea resin microballoons, polymethyl methacrylate balloons, and thermal expansion microcapsules. One kind of the hollow particles may be used alone, or two or more kinds of the hollow particles may be used in combination. In the present specification, in a case where two or more kinds of hollow particles are contained, the content of the hollow particles means a total amount thereof.

A particle diameter of the hollow particle is not particularly limited. From the viewpoint of maintaining the mechanical strength of the backing material layer 54 high while suppressing the viscosity of the curable resin composition to be low, for example, the particle diameter of the hollow particle is preferably 1 to 300 μm, more preferably 5 to 100 μm, and still more preferably 20 to 80 μm. The “particle diameter” of the hollow particle is synonymous with the “particle diameter” of the thermally conductive particle described above. That is, the “particle diameter” of the hollow particle is a number average particle diameter.

The other components may include a dispersant, a diluent, a colorant, a viscosity adjuster, a plasticizer, a curing accelerator, and the like. The content of the other components in the backing material layer 54 can be, for example, 10% to 20% by volume.

Examples of a preferred form of the backing material layer 54 include a form in which a resin including at least one kind of a polyurea resin, an epoxy resin having a polyurethane structure, or an epoxy resin having a polyetheramine structure, and thermally conductive particles are included, the resin has the above-described specific loss tangent, the backing material layer has the above-described specific storage elastic modulus, and the above-described hollow particles are included. In this form, regarding a content of each component in the backing material layer 54, the content of the resin is 25 to 50% by volume, and preferably 30 to 50% by volume. The content of the thermally conductive particles is preferably 30% to 60% by volume, more preferably 30% to 55% by volume, and still more preferably 30% to 50% by volume. The content of the hollow particles is preferably 10% to 20% by volume.

The backing material layer 54 is preferably formed of a curable resin composition. The curable resin composition preferably includes the thermally conductive particles and a resin component which is any of a combination of a polyisocyanate compound and a polyamine compound, a combination of an epoxy resin having a polyurethane structure and a polyamine compound, or a combination of an epoxy resin and a polyamine compound, in which at least one of the epoxy resin or the polyamine compound has a polyether structure.

### Method of Manufacturing Backing Material Layer

The curable resin composition constituting the backing material layer 54 can be prepared by a routine method. For example, the curable resin composition can be obtained by kneading the above-described thermally conductive particles, the resin component including at least one kind of a polyurea resin, an epoxy resin having a polyurethane structure, or an epoxy resin having a polyetheramine structure, and appropriately other components, as the components constituting the curable resin composition, with a kneading device such as a planetary centrifugal mixer, a kneader, a pressurization kneader, a Banbury mixer (continuous kneader), or two rolls. A mixing order of each component is not particularly limited. Kneading conditions are not particularly limited, and it is sufficient that the thermally conductive particles are dispersed in the resin component.

By curing the curable resin composition obtained in this way, the backing material layer 54 can be obtained. The curing conditions can be adjusted according to a chemical reaction of the resin component contained in the curable resin composition, and for example, the backing material layer 54 can be obtained by heating and curing at a specific temperature for a certain period of time.

The shape of the backing material layer 54 is not particularly limited. For example, the backing material layer may have a preferred shape by a mold during curing, or a desired backing material may be obtained by preparing a shect-shaped backing material and cutting this backing material by die cutting or the like. Since the backing material layer 54 of the present disclosure has excellent workability, it is possible to manufacture a desired backing material layer while suppressing an occurrence of deformation, breakage, and the like even in a case of performing dicing work into a desired shape at a pitch of μm order.

### Preferred Physical Properties of Filler

Considering that the physical properties of the backing material layer 54 change due to a change in temperature environment in which the ultrasonic endoscope 12 is placed, it is preferable that the filler 80 has a hardness higher than a hardness of the backing material layer 54. In this way, even in a case where a dimensional change of the backing material layer 54 may occur due to a change in temperature environment in which the ultrasonic endoscope 12 is placed, the dimensional change can be suppressed by the hardness of the backing material layer 54. Since the ultrasonic endoscope 12 has a very small diameter, a quality of the ultrasonic endoscope 12 can be stabilized by suppressing this dimensional change. In addition, since the hardness of the filler 80 is high, a fixing strength of the substrate 60 and the cable 100 can be improved.

The material of the filler 80 is not particularly limited. However, in order to provide heat radiation properties while providing a desired hardness as described above, for example, it is preferable that the filler 80 is configured to include an epoxy resin having a crosslinking density of 500 mol/m³ or more and 12,000 mol/m³ or less, preferably a crosslinking density of 3,000 mol/m³ or more and 10,000 mol/m³ or less.

As such an epoxy resin, an epoxy resin having an epoxy equivalent of 140 or less as illustrated in WO2023/054203A can be used. The filler 80 may be a filler in which an epoxy resin having an epoxy equivalent of 140 or less is cured alone, or a filler in which the epoxy resin is cured by reacting with a curing agent.

In the ultrasonic oscillator unit 46 configured as described above, in a case where each ultrasonic oscillator 48 of the ultrasonic oscillator array 50 is driven, and a voltage is applied to the electrode 52 of the ultrasonic oscillator 48, the piezoelectric body 49 vibrates to sequentially generate ultrasonic waves, and the irradiation of the ultrasonic waves is performed toward the observation target site of the subject. Then, as the plurality of ultrasonic oscillators 48 are sequentially driven by an electronic switch, such as a multiplexer, scanning with ultrasonic waves is performed in a scanning range along a curved surface on which the ultrasonic oscillator array 50 is disposed, for example, a range of about several tens mm from a center of curvature of the curved surface.

In a case where the echo signal reflected from the observation target site is received, the piezoelectric body 49 vibrates to generate a voltage and outputs the voltage as an electric signal corresponding to the received ultrasound echo to the ultrasound processor device 14. The electric signal is subjected to various kinds of signal processing in the ultrasound processor device 14 and then is displayed as an ultrasound image on the monitor 20.

### Modification Example of Endoscope

The characteristic physical properties of the backing material layer 54 and the filler 80 described above can be applied to ultrasonic endoscopes of all structures. For example, in the ultrasonic endoscope 12, the cable 100 may have a configuration in which the second region AR2 is not present (configuration in which the covering portion 101 is peeled off to expose the signal cable 110 on the distal end side, and the outer cover 102 is provided on the proximal end side with respect to an exposed portion of the signal cable 110), or may have a configuration in which the protrusion 102A is not present.

In addition, the signal cable 110 is not limited to the non-coaxial cable as shown in FIG. 5, and may be a coaxial cable, a twisted pair cable, or the like. In a case where the signal cable 110 is a coaxial cable, for example, a configuration is adopted in which a shield layer is provided around one signal line 112, and the shield layer is covered with an insulating layer. In a case where the signal cable 110 is a twisted pair cable, a configuration is adopted in which two signal lines 112 are twisted together.

In addition, although the ultrasonic endoscope 12 is a convex type, the technology of the present disclosure can also be applied to a radial type ultrasonic endoscope. The technology of the present disclosure can also be applied to a configuration in which the ultrasonic observation part is provided on the distal end side with respect to the endoscope observation part, in the radial type ultrasonic endoscope.

### EXAMPLES

Hereinafter, examples of the backing material layer 54 of the technology of the present disclosure will be described, but the backing material layer 54 is not limited to results thereof.

### 1 Preparation of Composition for Backing Material Layer

A composition for a backing material layer (curable resin composition) having the following composition was prepared.

### Polyurea Resin

A composition for a backing material layer was prepared by mixing 2.5 parts of metaxylene diisocyanate (manufactured by Tokyo Chemical Industry Co., Ltd.) as a polyisocyanate, 45 parts of a resin composition mixed at a ratio of 2 parts of ELASMER 250P (manufactured by Kumiai Chemical Industry Co., Ltd.) and 8 parts of ELASMER 650P (manufactured by Kumiai Chemical Industry Co., Ltd.) as a polyamine, and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### Epoxy Resin Having Polyurethane Structure

A composition for a backing material layer was prepared by mixing 10 parts of ADEKA RESIN EPU-11F (manufactured by ADEKA Corporation) as an epoxy resin having a polyurethane structure, 45 parts of a resin composition mixed at a ratio of 0.6 parts of 2,2,4-trimethylhexamethylenediamine (manufactured by Tokyo Chemical Industry Co., Ltd.) and 1.0 part of Gaskamine-328 (manufactured by Mitsubishi Gas Chemical Company, Inc.), and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### Epoxy Resin Having Polyetheramine Structure

A composition for a backing material layer was prepared by mixing 10 parts of jER828 (manufactured by Mitsubishi Chemical Corporation) as a bisphenol A epoxy resin, 45 parts of a resin composition mixed at a ratio of 4.5 parts of JEFFAMINE D400 (manufactured by Huntsman Corporation) and 6.0 parts of JEFFAMINE D2000 (manufactured by Huntsman Corporation) as a bifunctional polyether polyamine, and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### 2 Production, Measurement, and Evaluation of Backing Material Sheet

The composition for a backing material layer prepared as described above was poured into a square mold having one side of 30 mm and a desired depth and cured by heating at 80° C. for 18 hours and then at 150° C. for 1 hour to produce a square backing material sheet having one side of 30 mm and a desired thickness. The depth of the mold used and the thickness of the obtained sheet are 2 mm or 0.5 mm, respectively. The following measurements and evaluations were performed on the backing material sheet.

### (1) Measurement of Glass Transition Temperature

A loss tangent of the prepared 0.5 mm-thick backing material sheet was measured using a dynamic viscoelasticity analyzer (Vibron: DVA-225 (trade name), manufactured by IT Measurement Control Co., Ltd.) under conditions of a distance between grippers of 20 mm, a temperature rising rate of 2° C./min, a measurement temperature range of −150° C. to 250° C., and a frequency of 5 Hz, and a glass transition temperature was measured by obtaining the maximum value of the loss tangent.

The glass transition temperature of the backing material sheet composed of the composition for a backing material layer was in a range of 25 degrees to 35 degrees.

### (2) Measurement of Thermal Conductivity

The square backing material sheet having a thickness of 0.5 mm was cut into a strip shape having a width of 5 mm to prepare a test piece. The prepared test piece was measured by a laser flash method according to Japanese Industrial Standards (JIS) R 1611. The test piece formed of any of the compositions for a backing material layer exhibited a good value of a thermal conductivity of 1.0 W/m·K.

### (3) Measurement of Attenuation Rate

An intensity of a reflected echo was measured using a sing-around type acoustic velocity measurement apparatus (manufactured by Ultrasonic Engineering Co., Ltd., trade name “UVM-2 type”) based on a method described in a method of measuring an ultrasonic attenuation coefficient of a solid according to Japanese Industrial Standards (JIS) Z 2354 (2012). In the measurement, a measurement probe of 2 MHz was used in water at 23° C., and an attenuation rate was obtained from a difference in intensity of the reflected echo depending on the presence or absence of a measurement test piece used for measuring the acoustic velocity and a thickness of the measurement test piece. The test piece formed of any of the compositions for a backing material layer exhibited a good value of an attenuation rate of 4.0 dB/mm·MHz or more.

The above results show that, with the backing material formed of at least one kind of the polyurea resin, the epoxy resin having a polyurethane structure, or the epoxy resin having a polyetheramine structure, high heat radiation properties and a high attenuation rate inside the subject can be realized while realizing a glass transition temperature lower than the temperature inside the subject.

As described above, at least the following matters are described in the present specification.
(1)

An ultrasonic endoscope comprising: a distal end part including an ultrasound transmission/reception section and an imaging unit, in which the ultrasound transmission/reception section includes an ultrasonic oscillator and a backing material layer, and a glass transition temperature of the backing material layer is 45 degrees or lower.
(2)

The ultrasonic endoscope according to (1), in which the glass transition temperature is 10 degrees or higher.
(3)

The ultrasonic endoscope according to (2), in which the glass transition temperature is 40 degrees or lower.
(4)

The ultrasonic endoscope according to (3), in which the glass transition temperature is 20 degrees or higher.
(5)

The ultrasonic endoscope according to (4), in which the glass transition temperature is 25 degrees or higher and 35 degrees or lower.
(6)

The ultrasonic endoscope according to any one of (1) to (5), in which an accommodation portion that accommodates the ultrasound transmission/reception section and a cable connected to the ultrasonic oscillator, and a filler that fills a gap in the accommodation portion are provided at the distal end part, and a hardness of the filler is higher than a hardness of the backing material layer.
(7)

The ultrasonic endoscope according to (6), in which the filler is an epoxy resin having a crosslinking density of 500 mol/m³ or more and 12,000 mol/m³ or less.
(8)

The ultrasonic endoscope according to any one of (1) to (5), in which the backing material layer is configured to include at least one kind of a polyurea resin, an epoxy resin having a polyurethane structure, or an epoxy resin having a polyetheramine structure.
(9)

The ultrasonic endoscope according to (8), in which the backing material layer is configured to include a heat radiation filler.
(10)

The ultrasonic endoscope according to (9), in which a thermal conductivity of the heat radiation filler is 30 W/m·K or more.
(11)

The ultrasonic endoscope according to (10), in which the heat radiation filler includes at least one of aluminum oxide, tungsten oxide, silicon carbide, tungsten carbide, silicon nitride, boron nitride, or aluminum nitride.
(12)

The ultrasonic endoscope according to any one of (1) to (5), in which a thickness of the backing material layer is 0.5 mm or more and 1.5 mm or less.
(13)

The ultrasonic endoscope according to (12), in which a vibration frequency of the ultrasonic oscillator has a center frequency of 5 MHz or more and 12 MHz or less.
(14)

The ultrasonic endoscope according to any one of (1) to (5), in which the ultrasound transmission/reception section is provided on a distal end side with respect to the imaging unit.

### Explanation of References

10: ultrasonic examination system
12: ultrasonic endoscope
14: ultrasound processor device
16: endoscope processor device
18: light source device
20: monitor
21a: water supply tank
21b: suction pump
22: insertion part
24: operating part
26: universal cord
28a: air/water supply button
28b: suction button
29: angle knob
30: treatment tool insertion port
32A, 32B, 32C: connector
34a: air/water supply tube
34b: suction tube
36: ultrasonic observation part
38: endoscope observation part
40: distal end part
41: exterior member
42: bendable part
43: soft part
44: treatment tool outlet port
45: treatment tool channel
46: ultrasonic oscillator unit
47: laminate
48: ultrasonic oscillator
49: piezoelectric body
50: ultrasonic oscillator array
52: electrode
52a: individual electrode
52b: oscillator ground
54: backing material layer
60: substrate
60a, 60b, 60c: side
62: electrode pad
64: ground electrode pad
76: acoustic matching layer
78: acoustic lens
80: filler
82: observation window
84: objective lens
86: imaging element
88: illumination window
90: cleaning nozzle
100: cable
101: covering portion
102: outer cover
102A: protrusion
106: resin layer
108: second shield layer
110: signal cable
112: signal line
112a: conductor
112b: insulating layer
114: ground linc
116: first signal line bundle
116a: distal end
118: first shield layer
130: fixing part
410: accommodation space
410A: first space
410B: second space
AR1: first region
AR2: second region
AR3: third region

What is claimed is:

1. An ultrasonic endoscope comprising:
a distal end part including an ultrasound transmission and reception section and an imaging unit,
wherein the ultrasound transmission and reception section includes an ultrasonic oscillator and a backing material layer, and
a glass transition temperature of the backing material layer is 45 degrees or lower.

2. The ultrasonic endoscope according to claim 1,
wherein the glass transition temperature is 10 degrees or higher.

3. The ultrasonic endoscope according to claim 2,
wherein the glass transition temperature is 40 degrees or lower.

4. The ultrasonic endoscope according to claim 3,
wherein the glass transition temperature is 20 degrees or higher.

5. The ultrasonic endoscope according to claim 4,
wherein the glass transition temperature is 25 degrees or higher and 35 degrees or lower.

6. The ultrasonic endoscope according to claim 1,
wherein an accommodation portion that accommodates the ultrasound transmission and reception section and a cable connected to the ultrasonic oscillator, and a filler that fills a gap in the accommodation portion are provided at the distal end part, and
a hardness of the filler is higher than a hardness of the backing material layer.

7. The ultrasonic endoscope according to claim 2,
wherein an accommodation portion that accommodates the ultrasound transmission and reception section and a cable connected to the ultrasonic oscillator, and a filler that fills a gap in the accommodation portion are provided at the distal end part, and
a hardness of the filler is higher than a hardness of the backing material layer.

8. The ultrasonic endoscope according to claim 3,
wherein an accommodation portion that accommodates the ultrasound transmission and reception section and a cable connected to the ultrasonic oscillator, and a filler that fills a gap in the accommodation portion are provided at the distal end part, and
a hardness of the filler is higher than a hardness of the backing material layer.

9. The ultrasonic endoscope according to claim 4,
wherein an accommodation portion that accommodates the ultrasound transmission and reception section and a cable connected to the ultrasonic oscillator, and a filler that fills a gap in the accommodation portion are provided at the distal end part, and
a hardness of the filler is higher than a hardness of the backing material layer.

10. The ultrasonic endoscope according to claim 5,
wherein an accommodation portion that accommodates the ultrasound transmission and reception section and a cable connected to the ultrasonic oscillator, and a filler that fills a gap in the accommodation portion are provided at the distal end part, and
a hardness of the filler is higher than a hardness of the backing material layer.

11. The ultrasonic endoscope according to claim 6,
wherein the filler is an epoxy resin having a crosslinking density of 500 mol/m³ or more and 12,000 mol/m³ or less.

12. The ultrasonic endoscope according to claim 7,
wherein the filler is an epoxy resin having a crosslinking density of 500 mol/m³ or more and 12,000 mol/m³ or less.

13. The ultrasonic endoscope according to claim 8,
wherein the filler is an epoxy resin having a crosslinking density of 500 mol/m³ or more and 12,000 mol/m³ or less.

14. The ultrasonic endoscope according to claim 1,
wherein the backing material layer comprises at least one kind of a polyurea resin, an epoxy resin having a polyurethane structure, or an epoxy resin having a polyetheramine structure.

15. The ultrasonic endoscope according to claim 14,
wherein the backing material layer comprises a heat radiation filler.

16. The ultrasonic endoscope according to claim 15,
wherein a thermal conductivity of the heat radiation filler is 30 W/m·K or more.

17. The ultrasonic endoscope according to claim 16,
wherein the heat radiation filler comprises at least one of aluminum oxide, tungsten oxide, silicon carbide, tungsten carbide, silicon nitride, boron nitride, or aluminum nitride.

18. The ultrasonic endoscope according to claim 1,
wherein a thickness of the backing material layer is 0.5 mm or more and 1.5 mm or less.

19. The ultrasonic endoscope according to claim 18,
wherein a vibration frequency of the ultrasonic oscillator has a center frequency of 5 MHz or more and 12 MHz or less.

20. The ultrasonic endoscope according to claim 1,
wherein the ultrasound transmission and reception section is provided on a distal end side with respect to the imaging unit.
